(19) United States
(12) Patent Application Publication
(10) Pub. No.: US 2026/0269465 A1
(43) Pub. Date: Sep. 10, 2026

(54) ANTENNA WITH SELECTABLE GAIN

(71) Applicant: Insulet Corporation, Acton, MA (US)

(72) Inventors: Colan Ryan, Escondido, CA (US); Charles McCarthy, Bloomington, IL (US)

(21) Appl. No.: 19/553,015

(22) Filed: Feb. 27, 2026

Related U.S. Application Data
(60) Provisional application No. 63/766,463, filed on Mar. 4, 2025.

(51) Int. Cl. H01Q 1/44; H01Q 5/307; H03G 3/30

(52) U.S. Cl. CPC H01Q 1/44; H01Q 5/307; H03G 3/30

(57) ABSTRACT

A medical device such as an automated medicament delivery device for automated administration of medicament to a user-body or an analyte sensor such as a continuous glucose monitor for detecting analyte levels in a user-body are disclosed. The automated medicament delivery device includes an antenna, a communication chip, and a gain control circuit. The communication chip configured for operation of the antenna in at least two selectable modes including a high-gain mode and a low-gain mode. The high-gain mode and the low-gain mode of the antenna at least partially based on a state of a gain control circuit operatively coupling the communication chip with the antenna. The antenna, utilizing the gain control circuit, operable in the low-gain mode during performance of a pairing operation with a separate device and operable in the high-gain mode during ongoing external communication.

### CROSS-REFERENCE TO RELATED APPLICATIONS

This application claims the benefit under 35 U.S.C. § 119(e) of U.S. Provisional Patent Application Ser. No. 63/766,463, filed Mar. 4, 2025, the disclosure of which is hereby incorporated herein in its entirety by this reference.

### TECHNICAL FIELD

This disclosure relates generally to automated medicament delivery administration. Some embodiments relate to an antenna with selectable gain for automated medicament delivery systems and devices.

### BACKGROUND

Automated medicament delivery systems and devices (AMD, e.g., Automated Insulin Delivery (AID) device, without limitation) are often used to administer medicaments from a reservoir of the AMD to the body of a patient via a cannula inserted into the body to treat medical conditions (e.g., Type 1 Diabetes, without limitation).

The AMD often communicates with other devices, such as an analyte sensor and a controller (e.g., a handheld electronic computing device, such as a mobile device or dedicated handheld controller).

For communication with other devices, an AMD may pair with another device (e.g., exchange device information with the other device and register the device information received therefrom, without limitation) to link with the device and facilitate communication therewith. During a pairing process, the gain of the antenna of the AMD may be high, resulting in a large pairing range for the antenna during the pairing process. This large pairing range may allow the AMD to unintentionally communicate with devices that a user does not wish to pair the AMD with, which may enable a user to accidentally pair the AMD with the wrong device.

During testing, multiple AMDs may be programmed and tested simultaneously within close proximity to testing and programming equipment. The AMDs may interfere with one another and may require the utilization of shielded enclosures.

### BRIEF SUMMARY

In one or more illustrative embodiments, an automated medicament delivery device for automated administration of medicament to a user-body is disclosed. The automated medicament delivery device includes a delivery system, an antenna, a communication chip, a gain control circuit, one or more processors, and memory. The delivery system is configured to deliver medicament to the user-body. The communication chip configured for operation of the antenna in at least two selectable modes including a high-gain mode and a low-gain mode. A gain of the antenna in the low-gain mode at an operating frequency of the antenna is about a quarter or less of a gain of the antenna in the high-gain mode at the operating frequency. The high-gain mode and the low-gain mode of the antenna at least partially based on a state of a gain control circuit operatively coupling the communication chip with the antenna. The memory for storing instructions that, when executed by the one or more processors, enable the one or more processors to: operate the antenna utilizing the gain control circuit in the low-gain mode during performance of a pairing operation with a separate device; and operate the antenna utilizing the gain control circuit in the high-gain mode during ongoing external communication.

In one or more illustrative embodiments, a method for controlling an antenna of an automated medicament delivery device is disclosed. The method includes operating, with a communication chip, the antenna utilizing a gain control circuit in the low-gain mode during performance of a pairing operation with a separate device, the communication chip configured for operation of the antenna in at least two selectable modes including a high-gain mode and the low-gain mode, a gain of the antenna in the low-gain mode at an operating frequency of the antenna is at least a quarter of a gain of the antenna in the high-gain mode at the operating frequency, the high-gain mode and the low-gain mode of the antenna at least partially based on a state of the gain control circuit operatively coupling the communication chip with the antenna. The method also includes operating the antenna utilizing the gain control circuit in the high-gain mode during ongoing external communication.

### BRIEF DESCRIPTION OF THE DRAWINGS

For a detailed understanding of the disclosure, reference should be made to the following detailed description, taken in conjunction with the accompanying drawings, in which like elements have generally been designated with like numerals, and wherein:

FIG. 1 is a schematic diagram illustrating an automated medicament delivery device (or system);

FIG. 2 is a block diagram of an automated medicament delivery system for controlled administering of medicament, in accordance with one or more embodiments;

FIG. 3 is a perspective view of a PCB with communication equipment connected thereto, in accordance with one or more embodiments;

FIG. 4 is a detailed view of a gain control circuit of FIG. 3 illustrating current flow in a high-gain mode;

FIG. 5 is a detailed view of the gain control circuit of FIG. 3 illustrating current flow in a low-gain mode;

FIG. 6 is a detailed view of the gain control circuit of FIG. 3, in accordance with one or more embodiments;

FIG. 7 illustrates a performance plot for simulated results of a reflection magnitude of the antenna for a low-gain mode, high-gain mode, and a nominal configuration;

FIG. 8A illustrates a plot for simulated results of a radiation pattern of the realized gains of an antenna in a high-gain mode;

FIG. 8B illustrates a plot for simulated results of a radiation pattern of the realized gains of an antenna in a low-gain mode;

FIG. 9A illustrates an embodiment of a communication circuit including a gain control circuit with an impedance mismatch circuit, in accordance with one or more embodiments;

FIG. 9B illustrates an embodiment of a communication circuit including a gain control circuit with multiple impedance mismatch circuits, in accordance with one or more embodiments;

FIG. 10 illustrates a performance plot of a gain of the antenna in a low-gain mode and in a high-gain mode; and

FIG. 11 is a flowchart of a method for controlling an antenna of an automated medicament delivery device (or system), in accordance with one or more embodiments.

### DETAILED DESCRIPTION

In various embodiments, an automated medicament delivery device (or system) includes a gain control circuit including an assemblage of electronic elements configured for toggling an antenna between a high-gain mode and a low-gain mode. The gain control circuit may be external to a communication chip of the automated medicament delivery system or device, may be internal to the communication chip, or may be a combination thereof.

A gain of the antenna while operating in the low-gain mode is significantly lower than a gain of the antenna while operating in the high-gain mode. The automated medicament delivery device (or system) may be configured to start in (by default upon initial start-up or powering on, for example) or switch to and operate in the low-gain mode during a pairing process for pairing the automated medicament delivery device (or system) with a separate device, such as a controller or an analyte sensor.

The illustrations presented herein are not actual views of any system, device, or structure, or any component thereof, but are merely idealized representations, which are employed to describe various embodiments.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any system, device, or structure, when utilized in a conventional manner. Furthermore, these terms may refer to an orientation of elements of any system, device, or structure, as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

In this description the term “coupled” and derivatives thereof may be used to indicate that two elements co-operate or interact with each other. When an element is described as being “coupled” to another element, then the elements may be in direct physical or electrical contact or there may be intervening elements or layers present. In contrast, when an element is described as being “directly coupled” to another element, then there are no intervening elements or layers present. The term “connected” may be used in this description interchangeably with the term “coupled,” and has the same meaning unless expressly indicated otherwise or the context would indicate otherwise to a person having ordinary skill in the art.

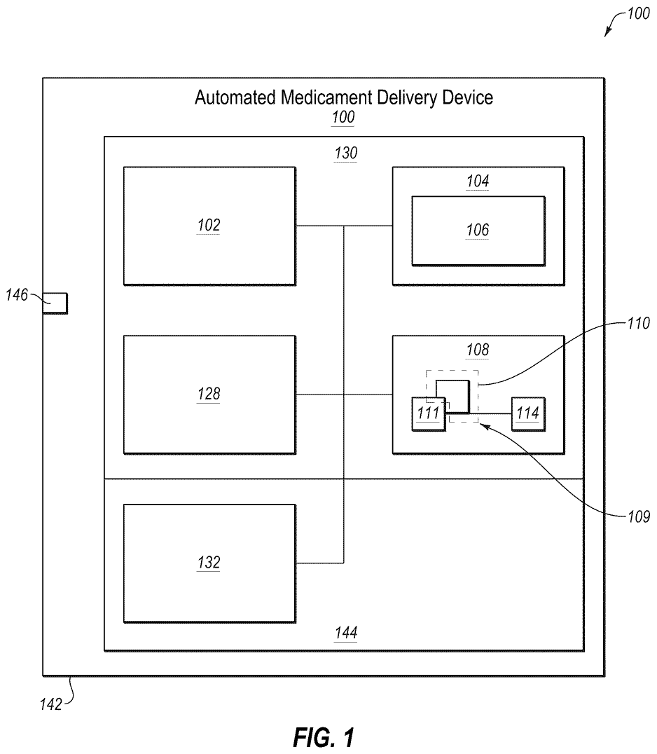

FIG. 1 is a schematic diagram illustrating an automated medicament delivery device (or system) 100 for automated administration of medicament to a user-body, in accordance with one or more embodiments.

In one or more embodiments, the automated medicament delivery device 100 may be capable of one or more operative modes of administration of medicament. Non-limiting examples of the one or more operative modes include: fully automated administration of medicament, partially automated administration of medicament, or manual administration of medicament. In one or more embodiments, the automated medicament delivery device 100 may be capable of alternating between multiple (e.g., two or more, without limitation) operative modes. As a non-limiting example, the automated medicament delivery device 100 may alternate between one or more of: fully automated operation, partially automated operation, and manual operation.

The automated medicament delivery device 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, hormones, and combinations or subcombinations of one or more of the foregoing, without limitation. The automated medicament delivery device 100 may also administer an amount of medicament at least partially based on user inputs (e.g., a user defined bolus amount or details related to a meal consumed or about to be consumed, such as number of carbohydrates, amount of fat, and/or amount of protein, without limitation).

Non-limiting examples of medicaments administrable by the automated medicament delivery device 100 include: insulin, glucagon, glucagon-like peptide-1 receptor agonist (GLP-1), glucose-dependent insulinotropic polypeptide (GIP), pramlintide, or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

The automated medicament delivery device 100 is configured to administer medicament to a user-body, such as subcutaneously into the user-body, without limitation, in accordance with one or more embodiments. In one or more embodiments, the automated medicament delivery device 100 may offer one or more operative modes for administration of medicament to a user-body. When operating in some of the operative modes, automated medicament delivery device 100 may administer medicament at least partially responsive to analyte values, including without limitation analyte values received from an analyte sensor. The analyte sensor is configured to obtain data related to one or more analytes within the user-body (“analyte data”). The analyte sensor may be an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G6 manufactured by Dexcom, without limitation).

When operating in some further operative modes, automated medicament delivery device 100 may administer medicament at least partially responsive to user input. When operating some yet further operative modes, automated medicament delivery device 100 may administer medicament at least partially responsive to analyte values and user input. Non-limiting examples of the one or more operative modes offered by automated medicament delivery device 100 include: fully automated administration of medicament, partially automated administration of medicament, or manual administration of medicament. When operating in an operative mode that includes manual administration of medicament, automated medicament delivery device 100 may administer medicament solely in response to a user input (e.g., delivers medicament in response to a user confirmation of delivery of medicament or in response to a user instruction to delivery medicament, without limitation). When operating in an operative mode that includes fully automated administration of medicament, automated medicament delivery device 100 may administer medicament solely in response to analyte values (e.g., delivers medicament in response to one or more analyte values, without limitation). When operating in an operative mode that includes partially automated administration of medicament, automated medicament delivery device 100 may administer medicament in response to analyte values and user input (e.g., delivers medicament in response to a user input and an analyte value, or alternately delivers medicament in response to a user input or in response to analyte values, without limitation). Medicament administration may include administration of a basal amount of medicament regularly delivered over a control interval (e.g., at a pre-determined basal rate, or a newly determined basal rate at each control interval, without limitation) to keep analyte levels stable and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and/or amount of protein, without limitation).

The automated medicament delivery device 100 includes a delivery system 132, one or more processors 102, memory 104, communication equipment 108, a PCB 130, and a power source 128. The automated medicament delivery device 100 may also include a housing 142 configured to enclose the various components of the automated medicament delivery device 100 and a chassis 144 configured to hold or support one or more components (e.g., one or more components of the delivery system 132) of the automated medicament delivery device 100. In one or more embodiments, the automated medicament delivery device 100, or portions thereof, may be a wearable device and may be secured to a user-body via an adhesive liner.

In various embodiments, the delivery system 132 is configured to cause an amount of medicament to move (e.g., flow, without limitation) toward and/or into a user-body. The delivery system 132 may deliver amounts of medicament at least partially responsive to requests. In various embodiments, instructions 106 of memory 104 may include instructions for determining and generating requests for delivery system 132. In various embodiments, instructions 106 may include instructions for determining one or more amounts of medicament, determining a timing for delivery of one or more amounts of medicament, and for generating one or more requests for delivery system 132 related to the same. When such instructions of instructions 106 are executed by one or more processors 102, the one or more processors 102 determine the amounts of medicament and timing of delivery, generate requests for the delivery system 132 at least partially based on the determined amounts and timing, and provide the requests to delivery system 132.

The communication equipment 108 is configured to facilitate communication (e.g., wireless communication, without limitation) of the automated medicament delivery device 100 with other devices, including without limitation communication between the automated medicament delivery device 100 and the analyte sensor and/or a controller (e.g., a dedicated electronic device, a smart phone, a tablet computer, a wearable device, without limitation). The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. The communication equipment 108 includes an antenna 114 configured to communicate with the other devices. The communication equipment 108 may also include a communication chip 111 (e.g., a BLUETOOTH® chip, without limitation) configured to control communication via the at least one antenna 114. The communication chip 111 may be an integrated circuit.

In various embodiments, the communication equipment 108 includes a communication circuit 109 operatively coupling the antenna 114 to the communication chip 111. The communication circuit 109 includes a feed line 121 and a gain control circuit 110. The feed line 121 connects the antenna 114 to the communication chip 111.

The gain control circuit 110 may integrate with the feed line 121 to operatively couple the communication chip 111 with the antenna 114. As will be described in further detail below, the communication chip 111 is configured for operation of the antenna 114 in at least two selectable modes including a high-gain mode and a low-gain mode, the high-gain mode and the low-gain mode of the antenna 114 at least partially based on a state of the gain control circuit 110.

The gain control circuit 110 may be configured to operate as a switching mechanism, togglable between different impedance states. The communication chip 111 may control the operation mode (e.g., the high-gain mode or the low-gain mode, without limitation) of the antenna 114 by controlling the impedance state of the gain control circuit 110. As will be described in further detail below, the gain control circuit 110 may include an assemblage of electronic elements operatively coupling the communication chip 111 and the antenna 114 via one or more pins of the communication chip 111, the communication chip 111, and/or other electronic elements (e.g., transmission lines, resistors, capacitors, inductors, and diodes, without limitation).

The antenna 114 may be an on-board antenna (e.g., on the PCB 130), an off-board antenna (e.g., off of the PCB 130), or an array of antennas (which may be any combination of on-board and off-board antennas). In various embodiments, the communication equipment 108 includes an Internet of Things (IOT) Subscriber Identity Module (SIM) card (e.g., a machine-to-machine SIM card, a Universal Integrated Circuit Card, without limitation).

The memory 104, one or more processors 102, and communication equipment 108 may be on and electrically connected via the PCB 130.

The power source 128 is configured to supply power to the delivery system 132 and the various electronic components, such as the one or more processors 102, memory 104, communication equipment 108, and the like. Power source 128 may be, as a non-limiting example, a power storage device (e.g., a battery, without limitation), a power inlet, a power regulator, or combination thereof.

The automated medicament delivery device 100 may include a pairing input 146 that is configured to provide a signal indicating that a user would like to pair the automated medicament delivery device 100 with another device (e.g., exchange device information with the other device and register the device information received therefrom, without limitation). The signal may be received at the one or more processors 102 and/or the communication chip 111 to initiate a pairing operation. The pairing input 146 may include a button or switch (e.g., a mechanical button/switch or a capacitive button/switch, without limitation).

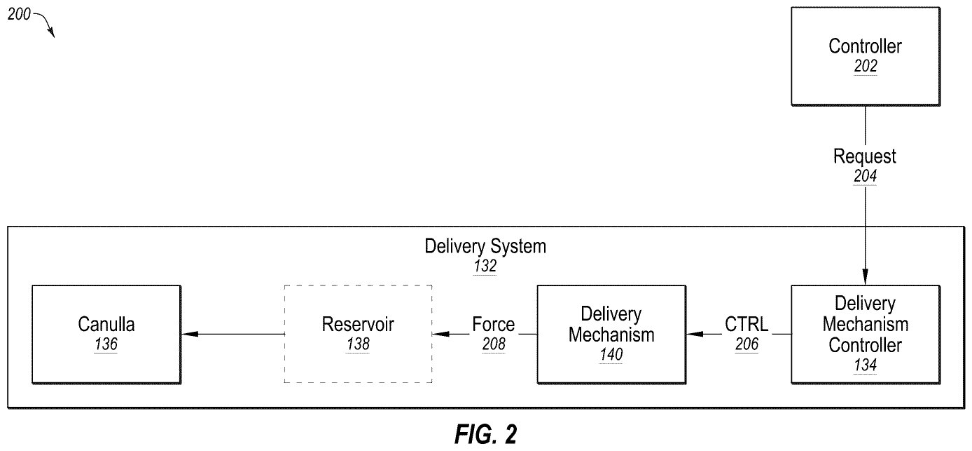

FIG. 2 is a block diagram of an automated medicament delivery system 200 for controlled administration of medicament to a user-body, in accordance with one or more embodiments.

Automated medicament delivery system 200 includes a delivery system 132 and an integrated controller 202 (“controller 202”). Delivery system 132 includes delivery mechanism controller 134, delivery mechanism 140, cannula 136, and reservoir 138. The reservoir 138, which holds the medicament, may be configured as a permanent fixture within the device or as a replaceable component, as a non-limiting example based on user needs or medicament refill practices. In FIG. 2, the block representing the reservoir 138, which stores the medicament, is outlined in dashed lines to indicate it may be either permanently within delivery system 132 or replaceable by a user within delivery system 132.

The controller 202 is configured to manage automated medicament delivery device 100 and, more generally, administration of medicament to a user-body. In one or more embodiments, controller 202 may be implemented by instructions 106 and one or more processors 102 of the automated medicament delivery device 100 of FIG. 1.

In various embodiments, controller 202 and delivery system 132 may be realized in different devices (e.g., controller 202 may be realized in a physically different device (or devices) than delivery system 132 is realized), or in the same device. When realized in different devices, functionality of controller 202 and delivery system 132 may be implemented, at least in part, by respective memory and one or more processors of their respective devices. When realized in a same device, functionality of controller 202 and delivery system 132 may be implemented, at least in part, by memory and one or more processors, respective memory and respective one or more processors, or a combination thereof. Non-limiting examples of devices in which controller 202, or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 202 may be configured to receive analyte data (e.g., from the analyte sensor, without limitation) including analyte values. In one or more embodiments, controller 202 may determine information about analytes within a user-body at least partially based on analyte data, for example, amounts, trends, distributions, without limitation. The controller 202 may analyze information about analytes in a user-body and may present the information and/or analysis to a patient, caregiver, or healthcare provider, as a non-limiting example, via an application (e.g., executing on a personal computer, smart phone, cloud server, or combinations thereof).

In various embodiments, the controller 202 may be configured to receive information from inputs from the patient or a caregiver (e.g., when the patient ate a meal or when the patient exercised, without limitation), and inputs from other electronic devices (e.g., information from a smartwatch, without limitation) and to utilize such information (e.g., process such information utilizing a control algorithm, without limitation) as discussed herein. For example, in various embodiments, controller 202 may utilize some or a totality of such information to determine amounts of medicament to administer and timing of administration of medicament. Further, controller 202 may also be configured to determine requests, including request to administer dose 204, and send those requests to the automated medicament delivery device 100.

In various embodiments, controller 202 may be configured to determine a target dose amount to administer to a user of automated medicament delivery system 200. Controller 202 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), doses of fast-acting or rapid-acting insulin, a current glucose value, and derivatives thereof without limitation. The timing and target dose amounts associated with requests generated by controller 202 may be governed by one or more control algorithms, discussed below.

Controller 202 may send a request to administer dose to delivery system 132, and more specifically, delivery mechanism controller 134.

The cannula 136 is insertable into a user-body (e.g., with a tip thereof positioned subcutaneously, without limitation) and is configured to provide medicament to a user-body (e.g., subcutaneously into the user-body, without limitation).

The reservoir 138 is configured to store and retain a medicament therein. As a non-limiting example, the reservoir 138 may be a hollow body, a flexible pouch, a chamber, a vial, without limitation. In various embodiments, reservoir 138 is a fluid reservoir for holding medicament and may be, as a non-limiting example, formed from the walls of a cartridge. In the cartridge example, delivery system 132 may include a chamber (i.e., a space or region defined within delivery system 132) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 138 will be greater in a pre-filled state than the volume in an exhausted state. Additionally or alternatively to the cartridge example, delivery system 132 may be a multi-part delivery device where one of the two parts includes the reservoir 138 and the other one of the two parts includes the delivery mechanism controller 134. The other one of the two parts may optionally further include controller 202. Either one of the two parts may optionally include delivery mechanism 140 (e.g., a piston pump or a reciprocating pump, without limitation). The one of the two parts that includes reservoir 138 may be disposable (i.e., a “disposable part”) and configured to be removably secured to the other part of automated medicament delivery system 200. When reservoir 138 is exhausted, the disposable part may be removed and a replacement part including a reservoir 138 optionally in a pre-filled state, may be installed.

Delivery mechanism 140 is configured to urge fluid in reservoir 138 toward an interface for dispensing fluid (interface not shown). In various embodiments, delivery mechanism 140 may be positioned adjacent to reservoir 138. The delivery mechanism 140 is configured to cause an amount of the medicament to be administered to the user-body by causing the amount to flow from the reservoir 138 toward and into a user-body via cannula 136, which is in fluidic communication with the reservoir 138. In various embodiments, delivery mechanism 140 may utilize any suitable mechanism to generate positive displacement or negative displacement to transfer amounts of medicament from reservoir 138 toward cannula 136 and a user-body.

For example, delivery mechanism 140 may apply a force to a piston free to move within reservoir 138, and via such a force, move the piston in a direction that urges fluid in reservoir 138 toward the aforementioned interface. In one or more embodiments, delivery mechanism 140 may include an electrical motor (e.g., an AC or DC motor) that produces a force to, directly or indirectly, move the piston to perform a delivery action. A delivery action dispenses at a predetermined rate or volume of medicament (i.e., a predictable amount of fluid over a predictable duration of time). The delivery mechanism 140 may be capable of multiple rates of delivery, and in one or more embodiments, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal 206 may be determined and generated by delivery mechanism controller 134 to correspond to a delivery action. A control signal 206 may also be referred to herein as a “command 206” or an “instruction 206.”

Delivery mechanism controller 134 may generate control signal(s) 206 corresponding to one or more delivery actions at least partially based on a request to administer dose 204 received from controller 202. Control signal 206 may include first control signals to cause delivery mechanism 140 to generate resultant force 208, and a second, different control signal(s) to cause drive delivery mechanism 140 to not or stop generating force 208. Utilizing control signals 206, delivery mechanism controller 134 may control a length of a duration of time that delivery mechanism 140 produces force 208 and applies it to dispense fluid from reservoir 138, and indirectly, an amount of fluid dispensed from reservoir 138.

When delivery mechanism controller 134 generates control signal(s) 206 in response to a request to administer dose 204 from controller 202, it may generate the control signal(s) 206 at least partially based on a value of a target dose amount included with, or indicated by, request to administer dose 204. One or more delivery actions may be utilized to dispense an amount fluid corresponding to a dose amount determined by controller 202. For example, a fluid amount dispensed according to a delivery action may be less than a dose amount. Generally speaking, the delivery mechanism 140 and delivery system 132 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more embodiments, delivery mechanism controller 134 may be configured to determine and generate feedback information about delivery actions, such as times of delivery actions and dispensed amounts, without limitation. Feedback information may be generated based on information generated by delivery mechanism 140 or by sensors utilized by delivery mechanism controller 134 to monitor operation of delivery mechanism 140 (sensors not depicted). For example, sensors to monitor mechanical movement, current consumption, a voltage profile of an electric motor, reservoir fluid amount, without limitation, may be utilized. Such information may be logged and provided to and stored at controller 202, without limitation, e.g., for later processing or reading, without limitation. For example, the logs can be processed to determine patterns that may be utilized to determine whether delivery system 132 is operating as expected (e.g., in a predictable manner, without limitation), and if a difference between actual and expected operation exceeds a threshold, delivery mechanism controller 134 may be updated (e.g., firmware, parameters, or both, of delivery mechanism controller 134 may be updated, without limitation) to compensate or correct for the difference. Additionally or alternatively to updating the firmware or parameters, in a multi-part system, one or more parts including delivery mechanism controller 134 or controller 202 may be indicated as needing replacement (e.g., an alarm or alert is generated at delivery system 132, automated medicament delivery system 200, a mobile device or computer in communication therewith, without limitation).

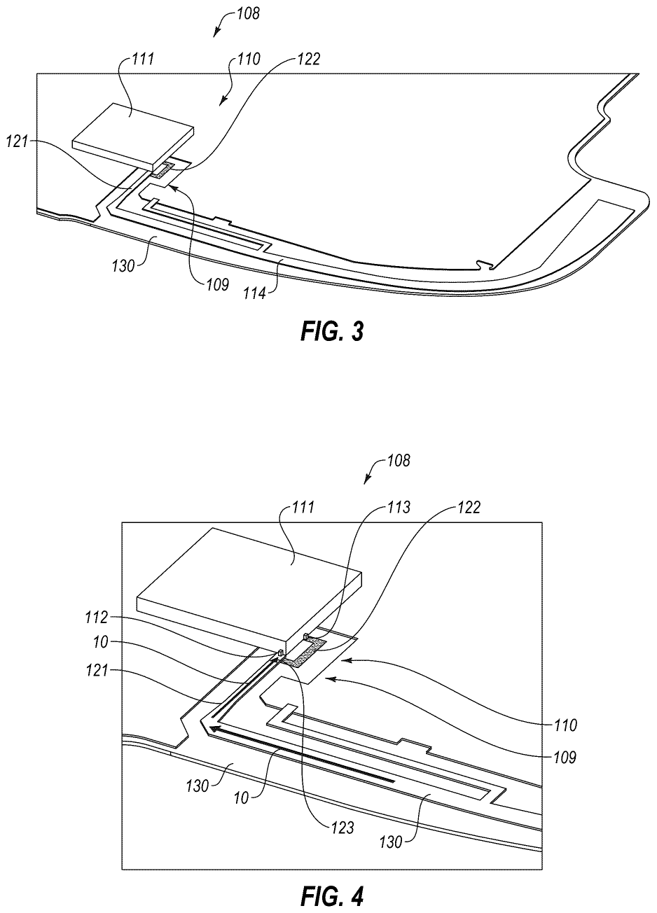

FIG. 3 is a perspective view of the PCB 130 with communication equipment 108 connected thereto, in accordance with one or more embodiments. Referring to FIG. 3, the communication equipment 108, and in particular, the communication chip 111, the antenna 114, and the communication circuit 109, is configured for dynamic control of the gain of the antenna 114. The communication circuit 109 operatively couples the antenna 114 to the communication chip 111. The communication circuit 109 includes the feed line 121 and the gain control circuit 110. The communication circuit 109 may also include a high-gain circuit (e.g., a circuit including one or more of the impedance mismatch circuits 117 described below, without limitation). The communication chip 111 may be configured (e.g., memory of the communication chip 111 includes instructions that, when executed, cause one or more processors of the communication chip 111, without limitation) to toggle operation of the antenna 114 between the high-gain mode and the low-gain mode via a state of the gain control circuit 110.

The high-gain mode may be a mode utilized for standard communication between the automated medicament delivery device 100 and other devices (e.g., an analyte sensor or a controller, without limitation), while the low-gain mode may be a mode utilized during pairing of the automated medicament delivery device 100 with another device. The gain and communication range of the antenna 114 operating in the low-gain mode may be significantly decreased compared to the gain and the communication range of the antenna 114 operating in the high-gain mode, such as at least half, a quarter, or an eighth of the gain and communication range of the antenna 114 in the high-gain mode. In various embodiments, the gain in the low-gain mode is at least 10 dB less than the gain in the high-gain mode, and may be at least 15 dB or 20 dB less. The communication range of the antenna 114 in the low-gain mode may be at least a third of the communication range of the antenna 114 in the high-gain mode. In various embodiments, the low-gain mode reduces the gain by about 20 dB and may reduce the communication range by about a factor of ten.

The gain control circuit 110 may be an internal circuit of the communication chip 111, an external circuit relative to the communication chip 111, or a combination thereof. The gain control circuit 110 may provide supplemental, circuit-level gain adjustment for the antenna 114, in addition to any internal gain control managed by the communication chip 111. In various embodiments, the supplemental, circuit level gain adjustment for the antenna 114 is achieved utilizing external impedance-based gain control, which operates alongside, and complementary with, any internal gain adjustments configured by the communication chip 111, allowing for additional flexibility in managing the operating range of the antenna 114.

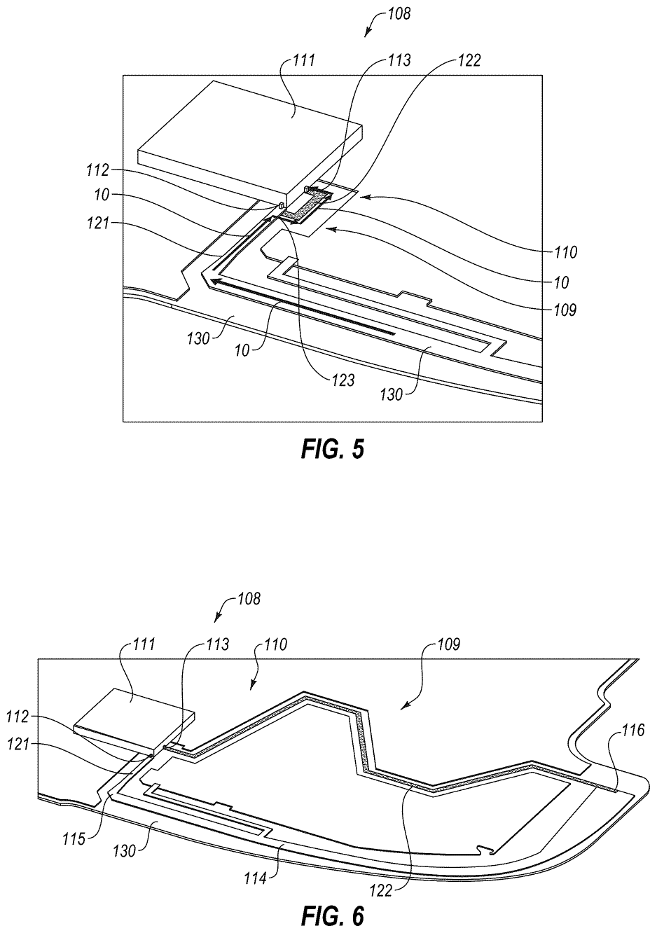

FIG. 4 is a detailed view of the gain control circuit 110 of FIG. 3 illustrating current flow in a high-gain mode. FIG. 5 is a detailed view of the gain control circuit 110 of FIG. 3 illustrating current flow in a low-gain mode. Referring to FIGS. 3-5, in various embodiments, the gain control circuit 110 includes a selection line 122 that integrates with the feed line 121 to operatively couple the antenna 114 to the communication chip 111. Each of the feed line 121 and the selection line 122 may be a trace on the PCB 130 and may connect to a pin of the communication chip 111 (e.g., a radio frequency (“RF”)) pin 112 or a General-purpose input/output (“GPIO” pin 113, without limitation). In various embodiments, the gain control circuit 110 includes a GPIO pin 113 with the selection line 122 connected thereto. The gain control circuit 110 may dynamically switch between high-gain mode and low-gain mode of the antenna 114 via activation or deactivation of the GPIO pin 113 of the communication chip 111, connected to the selection line 122, as discussed below.

In various embodiments, the feed line 121 connects the RF pin 112 to the antenna 114 and the selection line 122 connects the GPIO pin 113 to a location chosen from among: the feed line 121 between the connections to the antenna 114 and the RF pin 112 (FIGS. 3-5); and to the antenna 114 at a position offset from the connection between the antenna 114 and the feed line 121 (FIG. 6). The selection line 122 may connect to the GPIO pin 113 positioned on the communication chip 111 closest to the RF pin 112. The selection line 122 may connect to the feed line 121 closer to the RF pin 112 than to the antenna 114 (e.g., proximal to the connection between the feed line 121 and the RF pin 112, such as the closest possible connection point without interfering with the connection between the feed line 121 and the RF pin 112 or a connection point between the RF pin 112 and a high-gain circuit, without limitation) and may be arranged to be as short as possible based on available space for the feed line 121 and the selection line 122 on the PCB 130.

In various embodiments, the gain control circuit 110 is configured to alter impedance conditions along a connection between the communication chip 111 and antenna 114, which allows for the two or more selectable gain modes of the antenna 114. The impedance state of the GPIO pin 113 may set the impedance state of the selection line 122, and the impedance state of that combined circuit (i.e., the gain control circuit 110) sets the impedance state of the communication circuit 109 and, thus, the gain characteristic of the antenna 114.

In various embodiments, the RF pin 112 remains activated for communication in the high-gain mode and in the low-gain mode, and the GPIO pin 113 is deactivated in the high-gain mode and activated in the low-gain mode. The GPIO pin 113 may be deactivated by being put into a high-impedance state (e.g., utilizing a pull-up or pull-down resistor of the communication chip 111, without limitation). The high-impedance of the GPIO pin 113, which may be several hundred kiloohms, may appear as an open circuit at the connection point 123 between the selection line 122 and the feed line 121. While receiving, no current from the antenna 114 will flow into the open circuit and while transmitting, no current will flow from the communication chip 111 into the open circuit, thus effectively removing the selection line 122 from the active circuit. In this scenario, the antenna functions in a normal, high-gain state. While receiving, the current flows along arrows 10, along the antenna 114 and from the antenna 114 to the RF pin 112 via feed line 121. While transmitting, the current travels in the opposite direction of arrows 10, from the RF pin 112 to the antenna 114 via feed line 121 and along the antenna 114 away from the feed line 121.

The GPIO pin 113 may be activated by being put into a low impedance state (e.g., a few ohms, without limitation). The selection line 122 is added to the active circuit allowing current from the antenna 114 to flow into the selection line 122 while receiving and current from the communication chip 111 to flow into the selection line 122 while transmitting, which may short circuit the feed line 121, which may essentially short out the antenna 114 and result in a minimal gain/amount of radiation transferring between the antenna 114 and the communication chip 111. In this scenario, the current flows along arrows 10, along the antenna 114, down the feed line 121, and along the selection line 122, resulting in the antenna 114 functioning in a shorted, low-gain state.

The activation and deactivation of the GPIO pin 113 may be utilized to toggle the antenna 114 between the high-gain mode and the low-gain mode for both transmit and receive operations of the communication equipment 108.

FIG. 6 is a detailed view of the gain control circuit 110 of FIG. 3, in accordance with one or more embodiments. In various embodiments, the selection line 122 connects the GPIO pin 113 to the antenna 114 at a second connection point 116 offset from a first connection point 115 between the antenna 114 and the feed line 121 (e.g., to a distal end of the antenna 114 relative to a connection between the feed line 121 and the antenna 114, without limitation). A length of the selection line 122 may be about a fraction of a wavelength of the antenna 114, such as a one quarter wavelength interval (e.g., a one-quarter wavelength, such as about 20 mm a one-half wavelength, such as about 40 mm, a three-quarter wavelength, a one-to-one wavelength, a five-fourths wavelength, etc., without limitation) for the operating frequency of the antenna 114 (e.g., 2.45 GHz, without limitation). For example, a selection line 122 with a length that is one-half wavelength, the control may be identical to the above, with the GPIO pin 113 deactivated for the high-gain mode and activated for the low gain mode. For a selection line 122 with a length that is one-quarter wavelength, the mode of operation may be reversed: the GPIO pin 113 is deactivated for the low-gain mode and the GPIO pin 113 is activated for the high-gain mode.

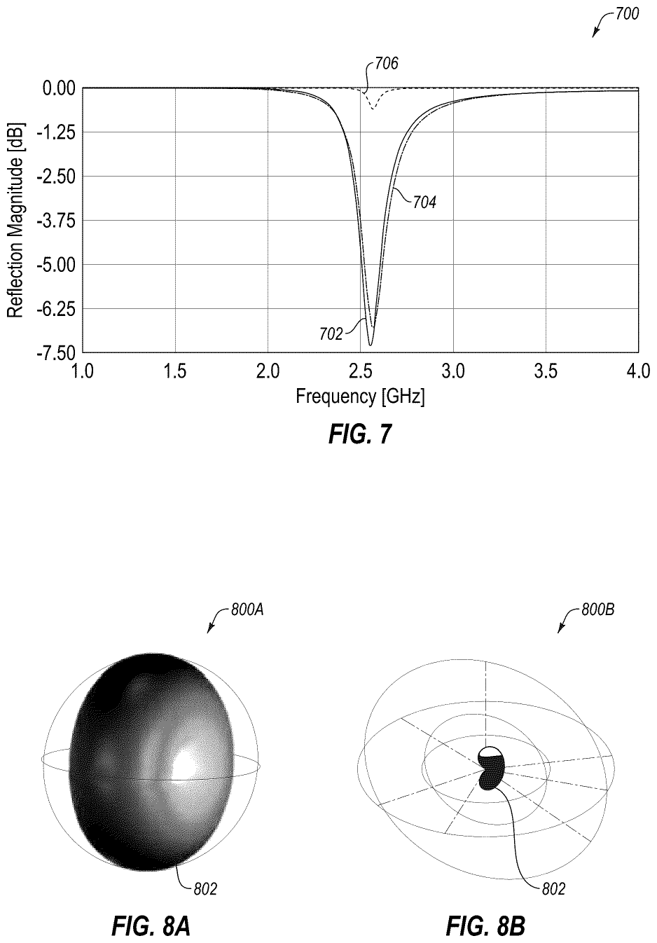

FIG. 7 illustrates a performance plot 700 for simulated results of a reflection magnitude of the antenna for a low-gain mode, high-gain mode, and a nominal configuration. Referring to FIG. 7, a nominal reflection magnitude 702 for a nominal configuration of communication equipment (i.e., an antenna configured without a gain control circuit), a high-gain mode reflection magnitude 704 for an antenna configured in a high-gain mode, and a low-gain mode reflection magnitude 706 for an antenna configured in a low-gain mode.

As illustrated in plot 700, the curve for the high-gain mode reflection magnitude 704 and the curve for the nominal reflection magnitude 702 nearly overlap, indicating substantially identical reflection magnitudes. In contrast, the curve for the low-gain mode reflection magnitude 706 is very different and close to 0 dB, indicating that most power is blocked from being received (in a receiving mode) by the communication chip or blocked from being transmitted (in a transmit mode).

FIG. 8A illustrates a plot 800A for simulated results of a radiation pattern of the realized gains 802 of an antenna in a high-gain mode. FIG. 8B illustrates a plot 800B for simulated results of a radiation pattern of the realized gains 802 of an antenna in a low-gain mode. Referring to FIG. 8A and FIG. 8B, the antenna operating in the low-gain mode has a very low gain (about −19 dB). The high-gain mode allows the antenna to function with good performance (about 1.2 dB), while the low-gain mode decreases the gain by approximately 20 dB. As illustrated, the low-gain mode decreases the possible communication range by about a factor of 10, which allows for intentionally reducing a range of an antenna when attempting to pair an automated medicament delivery device (or system) with another, nearby device. However, the range reduction may vary, depending on the precise environment that the automated medicament delivery device (or system) is operating in.

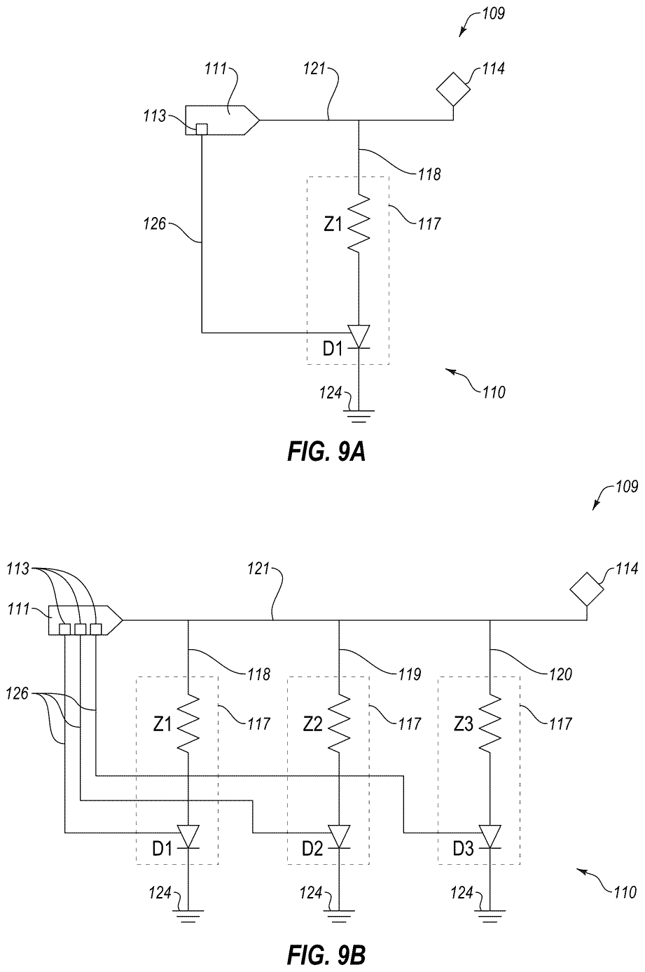

FIG. 9A illustrates an embodiment of a communication circuit 109 including a gain control circuit 110 with an impedance mismatch circuit 117, in accordance with one or more embodiments. FIG. 9B illustrates an embodiment of a communication circuit 109 including a gain control circuit with multiple impedance mismatch circuits 117, in accordance with one or more embodiments Referring to FIGS. 9A and 9B, the gain control circuit 110 is configured to integrate with the feed line 121 to operatively couple the antenna 114 to the communication chip 111 and to alter the impedance matching condition of the RF path (e.g., along the feed line 121 and the antenna 114, without limitation).

The gain control circuit 110 includes at least one impedance mismatch circuit 117 connected in parallel between the RF path and ground 124. Each impedance mismatch circuit 117 may connect to the RF path (e.g., to the feed line 121 or the antenna 114, without limitation) and to ground directly via a connector 118, 119, 120 or indirectly, such as with a capacitive or inductive coupling, without limitation. In various embodiment, each connector 118, 119, 120 is chosen from one or more of a transmission line, a short PCB trace, and a PCB pad, without limitation.

In various embodiments, the impedance mismatch circuit 117 includes a diode D1, D2, D3 and one or more impedance elements Z1, Z2, Z3. The diode D1, D2, D3 are operable to switch on and off to activate or deactivate the respective impedance elements Z1, Z2, Z3 of the respective impedance mismatch circuit 117 to cause an impedance mismatch and gain reduction in the communication circuit 109 (e.g., via resistance, reactance, or a combination thereof, without limitation). Each diode D1, D2, D3 may be controlled by voltage, current, or otherwise. The diodes D1, D2, D3 may be a Silicon-Controlled Rectifier (SCR) or a Thyristor, without limitation. In various embodiments, the diodes are replaced with other components or circuits that function to allow/block current flow (e.g., a transistor, without limitation).

Each impedance element Z1, Z2, Z3 may include resistors, capacitors, inductors, a transmission line with a length chosen to produce a preselected impedance, or a combination thereof chosen to produce the preselected impedance. In various embodiments, each impedance element Z1, Z2, Z3 includes a resistor. Each resistor may include a different resistance value. In some of these various embodiments, resistance is minimized to be close to zero ohms and a wire is used in place of a resistor.

While the gain control circuit 110 of the various embodiments of FIGS. 9A and 9B are illustrated as being external to the communication chip 111, in various embodiments, all or portions of the gain control circuit 110 (e.g., all or a portion of an impedance element Z1, Z2, Z3, and/or a diode D1, D2, D3, without limitation) may be internal to the communication chip 111.

In various embodiments, each diode D1, D2, D3 is operatively connected to a GPIO pin 113 and the communication chip 111 is configured to toggle operation of the antenna 114 between the high-gain mode and the low-gain mode of the via GPIO signals 126 sent to the respective diode D1, D2, D3. The diodes D1, D2, D3 may be switched on or off using the GPIO signals 126 from GPIO pins 113, which toggles operation of the antenna 114 between the high-gain mode and the low-gain mode. Each diode D1, D2, D3 may be connected to a different GPIO pin 113 for individual control thereof. One skilled in the art would understand the diode bias circuity required for utilizing the communication chip 111 to switch the diodes D1, D2, D3 on and off. In various embodiments including multiple impedance mismatch circuits 117 (e.g., 2, 3, 4, or more impedance mismatch circuits 117, without limitation), the communication circuit 109 may be configured to operate in multiple low-gain modes with each intermediate-gain modes configured to operate the antenna 114 with a gain lower than the gain of the high-gain mode. One or more impedance mismatch circuits 117 may be active for a given gain mode.

In these various embodiments, while the diodes D1, D2, D3 are inactive, the communication circuit 109 excludes the impedance element Z1, Z2, Z3 for each impedance mismatch circuit 117 of the gain control circuit 110, resulting in the antenna 114 functioning in a high-gain mode. In these various embodiments, while a diode D1, D2, D3 of the gain control circuit 110 is active, the gain control circuit 110 actively includes an impedance element Z1, Z2, Z3, which degrades the impedance matching, lowering the effective gain of the antenna 114 for a low-gain mode.

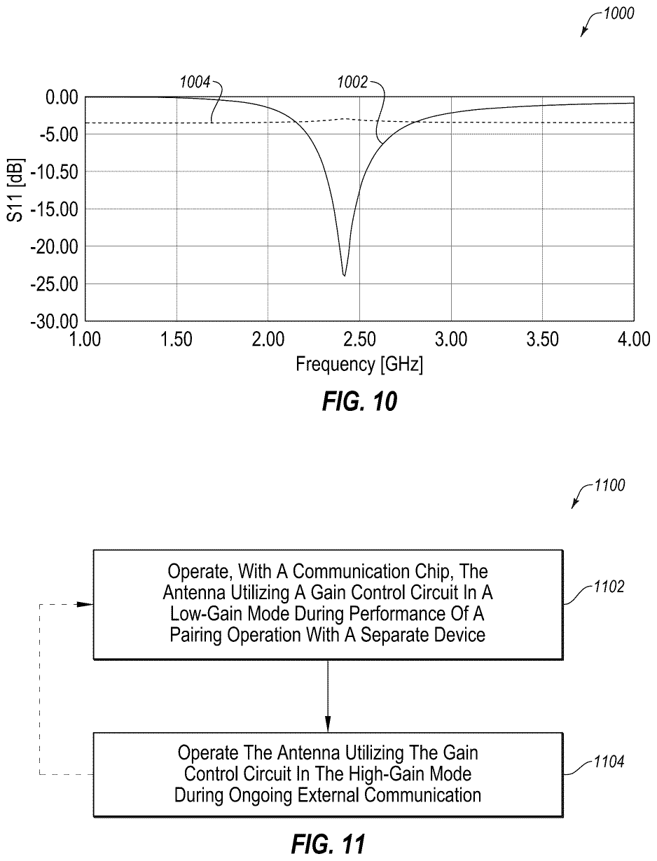

FIG. 10 illustrates a plot 1000 of reflection magnitude for simulated results of the antenna in a low-gain mode and in a high-gain mode utilizing a gain control circuit. Referring to FIG. 10, with the diodes inactive, the reflection magnitude curve 1002 for the high gain mode shows results identical or similar to a reflection magnitude curve for a nominal scenario, while with the diodes active, the reflection magnitude curve 1004 for the low gain mode shows a higher impedance mismatch, which lead to a reduced gain for select operating frequencies of the antenna 114 (e.g., 3 dB at 2.45 GHz according to the example simulation, without limitation).

With the various embodiments of the gain control circuit 110 of FIGS. 9A and 9B, specific low gain values may be achieved by controlling the degree of mismatch introduced in the RF path by one or more impedance mismatch circuits 117.

FIG. 11 is a flowchart of a method 1100 for controlling an antenna of an automated medicament delivery device (or system), in accordance with one or more embodiments. The method 1100 includes operating, with a communication chip, the antenna utilizing a gain control circuit in a low-gain mode (e.g., during performance of a pairing operation with a separate device, without limitation) at act 1102. The communication chip may be configured for operation of the antenna in at least two selectable modes including a high-gain mode and the low-gain mode where a gain of the antenna in the low-gain mode at an operating frequency of the antenna is about a quarter or less of a gain of the antenna in the high-gain mode at the operating frequency. The high-gain mode and the low-gain mode of the antenna may be at least partially based on a state of the gain control circuit operatively coupling the communication chip with the antenna. The gain control circuit may be any gain control circuit disclosed herein. Act 1102 may include toggling the gain control circuit from the high-gain mode to the low-gain mode responsive to initiating the pairing operation (e.g., receiving a signal for initiating the pairing operation, without limitation). The pairing operation may include processes from any pairing operation (e.g., a BLUETOOTH® pairing operation, without limitation) known in the art. Toggling the gain control circuit from the high-gain mode to the low-gain mode may be one of the initial processes of the pairing operation (e.g., prior to sending or receiving device information, without limitation).

The method 1100 also includes operating the antenna utilizing the gain control circuit in the high-gain mode during ongoing external communication at act 1104. Act 1104 may be performed after completing the pairing operation. Act 1104 may include toggling the gain control circuit from the low-gain mode to the high-gain mode responsive to completing the pairing operation (e.g., after sending and receiving device information with the separate device and registering the device information of the separate device, without limitation). Toggling the gain control circuit from the low-gain mode to the high-gain mode may be one of the final processes of the pairing operation (e.g., after sending and receiving the device information, without limitation).

The automated medicament delivery device (or system) may perform multiple pairing operations throughout use. In various embodiments, the method 1100 may include returning to act 1102 (e.g., upon selection of a pairing mode, without limitation) indicated by a dashed line in FIG. 11.

Toggling the gain control circuit between the high-gain mode and the low-gain mode may include activating/deactivating one or more GPIO pins of a communication chip and/or activating/deactivating diodes of the gain control circuit.

In various embodiments, the method includes operating the antenna utilizing the gain control circuit in a secondary low-gain mode where the gain of the antenna in the secondary low-gain mode at the operating frequency of the antenna is between the gain of the antenna in the high-gain mode at the operating frequency and the gain of the antenna in the low-gain mode at the operating frequency. The secondary low-gain mode may be utilized as part of the normal communication process when very low power is required (e.g., when the transmitter and the receiver are very close to each other, or to reduce interference with other devices, without limitation).

The method may be implemented by memory for including instructions that, when executed, enable the one or more processors (e.g., memory and one or more processors integrated into a communication chip 111, memory 104, one or more processors 102, or a combination thereof) to perform the acts of the method 1100.

In various embodiments, the automated medicament delivery device (or system) may be set to operate the antenna utilizing the gain control circuit in the low-gain mode for an extended period of time outside of a pairing operation, such as for testing the automated medicament delivery device (or system) or while operating in areas where interference with other devices may regularly occur, without limitation. The operation of the antenna utilizing the gain control circuit in the low-gain mode outside of a pairing operation may be initialized by an option in a user interface of a controller, by a preselected sequence of button presses on the automated medicament delivery device (or system) (e.g., a predetermined pattern or multiple buttons pressed simultaneously, without limitation), by initiating a pairing operation in a test environment, or by initiating a test of the automated medicament delivery device (or system), without limitation. By operating the automated medicament delivery device (or system) utilizing the gain control circuit in the low-gain mode outside of pairing, multiple automated medicament delivery devices (or systems) may be tested simultaneously in close proximity without interfering with one another and without the need for placing each of the automated medicament delivery device (or system) within a shielded enclosure. Further, multiple automated medicament delivery devices (or system) may be able to operate in close proximity without interfering with communication (e.g., in a clinical or hospital setting, without limitation).

While embodiments of the gain control circuit and methods of use thereof discussed herein are described in connection with automated medicament delivery systems and devices, such a gain control circuit may also be utilized within other devices, including medical devices (e.g., analyte sensors, such as continuous glucose monitors, being paired with a controller or an automated medicament delivery system, without limitation), optionally with modifications that would be apparent to a person having ordinary skill in the art, such as an analyte sensor or other wearable device, without limitation.

Non-limiting illustrative embodiments of this disclosure may include:

Embodiment 1: An automated medicament delivery device for automated administration of medicament to a user-body, the automated medicament delivery device comprising: a delivery system configured to deliver medicament to the user-body; an antenna; a communication chip configured for operation of the antenna in at least two selectable modes including a high-gain mode and a low-gain mode, a gain of the antenna in the low-gain mode at an operating frequency of the antenna is about a quarter or less of a gain of the antenna in the high-gain mode at the operating frequency, the high-gain mode and the low-gain mode of the antenna at least partially based on a state of a gain control circuit operatively coupling the communication chip with the antenna; one or more processors; and memory for storing instructions that, when executed by the one or more processors, enable the one or more processors to: operate the antenna utilizing the gain control circuit in the low-gain mode during performance of a pairing operation with a separate device; and operate the antenna utilizing the gain control circuit in the high-gain mode during ongoing external communication.

Embodiment 2: The automated medicament delivery device according to Embodiment 1, wherein the instructions that, when executed, further enable the one or more processors to: toggle the operation of the antenna from the high-gain mode to the low-gain mode responsive to initiating the pairing operation; and toggle the operation of the antenna from the low-gain mode to the high-gain mode responsive to completing the pairing operation.

Embodiment 3: The automated medicament delivery device according to any of Embodiments 1 and 2, wherein toggling the operation of the antenna between the high-gain mode and the low-gain mode includes activating or deactivating a General-purpose input/output pin (GPIO pin) of the communication chip, wherein a Radio Frequency pin (RF pin) of the communication chip connects to the antenna via a feed line.

Embodiment 4: The automated medicament delivery device according to any of Embodiments 1 through 3, wherein toggling the operation of the antenna between the high-gain mode and the low-gain mode includes activating or deactivating diodes of the gain control circuit.

Embodiment 5: The automated medicament delivery device according to any of Embodiments 1 through 4, wherein the memory and the one or more processors are integrated into the communication chip.

Embodiment 6: The automated medicament delivery device according to any of Embodiments 1 through 5, further comprising a feed line connecting the antenna to the RF pin, and wherein the gain control circuit is chosen from among: a first circuit comprising a first selection line connecting a GPIO pin to the feed line between connections of the feed line to the antenna and the RF pin; and a second circuit comprising a second selection line connecting the GPIO pin to the antenna offset from a connection between the antenna and the feed line.

Embodiment 7: The automated medicament delivery device according to any of Embodiments 1 through 6, wherein the GPIO pin is positioned on the communication chip closest to the RF pin than other GPIO pins of the communication chip.

Embodiment 8: The automated medicament delivery device according to any of Embodiments 1 through 7, wherein the first selection line connects to the feed line proximal to the connection between the feed line and the RF pin.

Embodiment 9: The automated medicament delivery device according to any of Embodiments 1 through 8, wherein a length of the second selection line is a one-quarter wavelength interval for the operating frequency of the antenna.

Embodiment 10: The automated medicament delivery device according to any of Embodiments 1 through 9, further comprising: a feed line connecting the antenna to the communication chip, the feed line and the antenna defining an RF path, and wherein the gain control circuit comprises at least one impedance mismatch circuit connected between the RF path and ground, each of the at least one impedance mismatch circuit comprising an impedance mismatch element and a diode operable to switch on and off to activate or deactivate the impedance mismatch element, the impedance mismatch element chosen from among one or more of a resistor, a capacitor, an inductor, and a transmission line with a length chosen to produce a preselected impedance.

Embodiment 11: The automated medicament delivery device according to any of Embodiments 1 through 10, wherein the gain control circuit is at least partially integrated into the communication chip.

Embodiment 12: A method for controlling an antenna of an automated medicament delivery device, the method comprising: operating, with a communication chip, the antenna utilizing a gain control circuit in a low-gain mode during performance of a pairing operation with a separate device, the communication chip configured for operation of the antenna in at least two selectable modes including a high-gain mode and the low-gain mode, a gain of the antenna in the low-gain mode at an operating frequency of the antenna is at least a quarter of a gain of the antenna in the high-gain mode at the operating frequency, the high-gain mode and the low-gain mode of the antenna at least partially based on a state of the gain control circuit operatively coupling the communication chip with the antenna; and operating the antenna utilizing the gain control circuit in the high-gain mode during ongoing external communication.

Embodiment 13: The method according to Embodiment 12, further comprising: toggling the operation of the antenna from the high-gain mode to the low-gain mode responsive to initiating the pairing operation; and toggling the operation of the antenna from the low-gain mode to the high-gain mode responsive to completing the pairing operation.

Embodiment 14: The method according to any of Embodiments 12 and 13, wherein toggling the operation of the antenna between the high-gain mode and the low-gain mode includes activating or deactivating a General-purpose input/output pin (GPIO pin) of a communication chip, wherein a Radio Frequency pin (RF pin) of the communication chip connects to the antenna via a feed line.

Embodiment 15: The method according to any of Embodiments 12 through 14, wherein the gain control circuit is chosen from among: a first circuit comprising a first selection line connecting a GPIO pin to the feed line between connections of the feed line to the antenna and the RF pin; and a second circuit comprising a second selection line connecting the GPIO pin to the antenna offset from a connection between the antenna and the feed line.

Embodiment 16: The method according to any of Embodiments 12 through 15, wherein the GPIO pin is positioned on the communication chip closest to the RF pin than other GPIO pins of the communication chip.

Embodiment 17: The method according to any of Embodiments 12 through 16, wherein the first selection line connects to the feed line proximal to a connection between the feed line and the RF pin.

Embodiment 18: The method according to any of Embodiments 12 through 17, wherein a length of the second selection line is a one-quarter wavelength interval for the operating frequency of the antenna.

Embodiment 19: The method according to any of Embodiments 12 through 18, wherein toggling the operation of the antenna between the high-gain mode and the low-gain mode includes activating or deactivating diodes of the gain control circuit.

Embodiment 20: The method according to any of Embodiments 12 through 19, wherein a feed line connects the antenna to the communication chip, a high-gain circuit integrates with the feed line and the gain control circuit to operatively couple the antenna to the communication chip, and wherein the gain control circuit comprises: a first transmission line, connected to the feed line between the communication chip and the high-gain circuit, including a first low gain resistor and a first diode thereon; and a second transmission line, connected to the feed line between the antenna and the high-gain circuit, including a second low gain resistor and a second diode thereon.

Embodiment 21: An automated medicament delivery device for automated administration of medicament to a user-body, the automated medicament delivery device comprising: a delivery system configured to deliver medicament to the user-body; an antenna; a communication chip comprising a Radio Frequency (RF) pin and a General-purpose input/output(GPIO) pin, the communication chip configured for operation of the antenna in at least two selectable modes including a high-gain mode and a low-gain mode, a gain of the antenna in the low-gain mode at an operating frequency of the antenna is about a quarter or less of a gain of the antenna in the high-gain mode at the operating frequency, the high-gain mode and the low-gain mode of the antenna at least partially based on a state of a gain control circuit; a feed line connecting the antenna to the RF pin; the gain control circuit chosen from among: a first circuit comprising a first selection line connecting the GPIO pin to the feed line between connections to the antenna and the RF pin; and a second circuit comprising a second selection line connecting the GPIO pin to the antenna offset from a connection between the antenna and the feed line.

Embodiment 22: The automated medicament delivery device according to Embodiment 21, the communication chip is configured to: operate the antenna utilizing the gain control circuit in the low-gain mode during performance of a pairing operation with a separate device; and operate the antenna utilizing the gain control circuit in the high-gain mode during ongoing external communication, wherein a gain of the antenna operating in the low-gain mode at an operating frequency of the antenna is at least a quarter of a gain of the antenna operating in the high-gain mode at the operating frequency.

Embodiment 23: An automated medicament delivery device for automated administration of medicament to a user-body, the automated medicament delivery device comprising: a delivery system configured to deliver medicament to the user-body; an antenna; a communication chip configured for operation of the antenna in at least two selectable modes including a high-gain mode and a low-gain mode, a gain of the antenna in the low-gain mode at an operating frequency of the antenna is less than a gain of the antenna in the high-gain mode at the operating frequency, the high-gain mode and the low-gain mode of the antenna at least partially based on a state of a gain control circuit; and a feed line connecting the antenna to the communication chip the feed line and the antenna defining an RF path the gain control circuit comprising: at least one impedance mismatch circuit connected between the RF path and ground, each of the at least one impedance mismatch circuit comprising an impedance mismatch element and a diode operable to switch on and off to activate or deactivate the impedance element, the impedance mismatch element chosen from among one or more of a resistor, a capacitor, an inductor, and a transmission line with a length chosen to produce a preselected impedance.

Embodiment 24: The automated medicament delivery device according to Embodiment 23, wherein the first diode and the second diode are operatively connected to one or more General-purpose input/output (GPIO) pins and the communication chip is configured to toggle operation of the antenna between the high-gain mode and the low-gain mode of the via GPIO signals sent to the first diode and the second diode.

Embodiment 25: The automated medicament delivery device according to any of Embodiments 23 and 24, wherein the communication chip is configured to: operate the antenna utilizing the gain control circuit in the low-gain mode during performance of a pairing operation with a separate device; and operate the antenna utilizing the gain control circuit in the high-gain mode during ongoing external communication.

Embodiment 26: The automated medicament delivery device according to any of Embodiments 23 through 25, wherein a gain of the antenna operating in the low-gain mode at an operating frequency of the antenna is about a quarter or less of a gain of the antenna operating in the high-gain mode at the operating frequency.

Embodiment 27: A first medical device configured to be paired to a second device, the first medical device comprising: an antenna; a communication chip configured for operation of the antenna in at least two selectable modes including a high-gain mode and a low-gain mode, a gain of the antenna in the low-gain mode at an operating frequency of the antenna is about a quarter or less of a gain of the antenna in the high-gain mode at the operating frequency, the high-gain mode and the low-gain mode of the antenna at least partially based on a state of a gain control circuit operatively coupling the communication chip with the antenna; one or more processors; and memory for storing instructions that, when executed by the one or more processors, enable the one or more processors to: operate the antenna utilizing the gain control circuit in the low-gain mode during performance of a pairing operation with the second device; and operate the antenna utilizing the gain control circuit in the high-gain mode during ongoing external communication.

The embodiments described above and illustrated in the accompanying drawings do not limit the scope of the disclosure, which is encompassed by the scope of the appended claims and their legal equivalents. Any equivalent embodiments are within the scope of this disclosure. Indeed, various modifications, in addition to those shown and described herein, such as alternate useful combinations of the elements described, will become apparent to those skilled in the art from the description. Such modifications and embodiments also fall within the scope of the appended claims and equivalents.

What is claimed is:

1. An automated medicament delivery device for automated administration of medicament to a user-body, the automated medicament delivery device comprising:
a delivery system configured to deliver medicament to the user-body;
an antenna;
a communication chip configured for operation of the antenna in at least two selectable modes including a high-gain mode and a low-gain mode, a gain of the antenna in the low-gain mode at an operating frequency of the antenna is about a quarter or less of a gain of the antenna in the high-gain mode at the operating frequency, the high-gain mode and the low-gain mode of the antenna at least partially based on a state of a gain control circuit operatively coupling the communication chip with the antenna;
one or more processors; and
memory for storing instructions that, when executed by the one or more processors, enable the one or more processors to:
operate the antenna utilizing the gain control circuit in the low-gain mode during performance of a pairing operation with a separate device; and
operate the antenna utilizing the gain control circuit in the high-gain mode during ongoing external communication.

2. The automated medicament delivery device of claim 1, wherein the instructions that, when executed, further enable the one or more processors to:
toggle the operation of the antenna from the high-gain mode to the low-gain mode responsive to initiating the pairing operation; and
toggle the operation of the antenna from the low-gain mode to the high-gain mode responsive to completing the pairing operation.

3. The automated medicament delivery device of claim 2, wherein toggling the operation of the antenna between the high-gain mode and the low-gain mode includes activating or deactivating a General-purpose input/output pin (GPIO pin) of the communication chip, wherein a Radio Frequency pin (RF pin) of the communication chip connects to the antenna via a feed line.

4. The automated medicament delivery device of claim 2, wherein toggling the operation of the antenna between the high-gain mode and the low-gain mode includes activating or deactivating diodes of the gain control circuit.

5. The automated medicament delivery device of claim 1, wherein the memory and the one or more processors are integrated into the communication chip.

6. The automated medicament delivery device of claim 1, further comprising a feed line connecting the antenna to a Radio Frequency pin (RF pin), and wherein the gain control circuit is chosen from among:
a first circuit comprising a first selection line connecting a GPIO pin to the feed line between connections of the feed line to the antenna and the RF pin; and
a second circuit comprising a second selection line connecting the GPIO pin to the antenna offset from a connection between the antenna and the feed line.

7. The automated medicament delivery device of claim 6, wherein the GPIO pin is positioned on the communication chip closest to the RF pin than other GPIO pins of the communication chip.

8. The automated medicament delivery device of claim 6, wherein the first selection line connects to the feed line proximal to the connection between the feed line and the RF pin.

9. The automated medicament delivery device of claim 6, wherein a length of the second selection line is a one-quarter wavelength interval for the operating frequency of the antenna.

10. The automated medicament delivery device of claim 1, further comprising: a feed line connecting the antenna to the communication chip, the feed line and the antenna defining an RF path, and wherein the gain control circuit comprises:
at least one impedance mismatch circuit connected between the RF path and ground, each of the at least one impedance mismatch circuit comprising an impedance mismatch element and a diode operable to switch on and off to activate or deactivate the impedance mismatch element, the impedance mismatch element chosen from among one or more of a resistor, a capacitor, an inductor, and a transmission line with a length chosen to produce a preselected impedance.

11. The automated medicament delivery device of claim 1, wherein the gain control circuit is at least partially integrated into the communication chip.

12. A method for controlling an antenna of an automated medicament delivery device, the method comprising:
operating, with a communication chip, the antenna utilizing a gain control circuit in a low-gain mode during performance of a pairing operation with a separate device, the communication chip configured for operation of the antenna in at least two selectable modes including a high-gain mode and the low-gain mode, a gain of the antenna in the low-gain mode at an operating frequency of the antenna is at least a quarter of a gain of the antenna in the high-gain mode at the operating frequency, the high-gain mode and the low-gain mode of the antenna at least partially based on a state of the gain control circuit operatively coupling the communication chip with the antenna; and
operating the antenna utilizing the gain control circuit in the high-gain mode during ongoing external communication.

13. The method of claim 12, further comprising:
toggling the operation of the antenna from the high-gain mode to the low-gain mode responsive to initiating the pairing operation; and
toggling the operation of the antenna from the low-gain mode to the high-gain mode responsive to completing the pairing operation.

14. The method of claim 13, wherein toggling the operation of the antenna between the high-gain mode and the low-gain mode includes activating or deactivating a General-purpose input/output pin (GPIO pin) of a communication chip, wherein a Radio Frequency pin (RF pin) of the communication chip connects to the antenna via a feed line.

15. The method of claim 14, wherein the gain control circuit is chosen from among:
a first circuit comprising a first selection line connecting a GPIO pin to the feed line between connections of the feed line to the antenna and the RF pin; and
a second circuit comprising a second selection line connecting the GPIO pin to the antenna offset from a connection between the antenna and the feed line.

16. The method of claim 15, wherein the GPIO pin is positioned on the communication chip closest to the RF pin than other GPIO pins of the communication chip.

17. The method of claim 15, wherein the first selection line connects to the feed line proximal to a connection between the feed line and the RF pin.

18. The method of claim 15, wherein a length of the second selection line is a one-quarter wavelength interval for the operating frequency of the antenna.

19. The method of claim 13, wherein toggling the operation of the antenna between the high-gain mode and the low-gain mode includes activating or deactivating diodes of the gain control circuit.

20. The method of claim 19, wherein a feed line connects the antenna to the communication chip, a high-gain circuit integrates with the feed line and the gain control circuit to operatively couple the antenna to the communication chip, and wherein the gain control circuit comprises:
a first transmission line, connected to the feed line between the communication chip and the high-gain circuit, including a first low gain resistor and a first diode thereon; and
a second transmission line, connected to the feed line between the antenna and the high-gain circuit, including a second low gain resistor and a second diode thereon.
